(19) United States
(12) Patent Application Publication
(10) Pub. No.: US 2009/0124892 A1
(43) Pub. Date: May 14, 2009

(54) Method for measuring cardiac perfusion in a patient and CT system for carrying out the method

(76) Inventors: Herbert Bruder, Hochstadt (DE); Karl Stierstorfer, Erlangen (DE); Carsten Thierfelder, Pinzberg (DE)

Correspondence Address: HARNESS, DICKEY & PIERCE, P.L.C., P.O.BOX 8910, RESTON, VA, 20195, US

(21) Appl. No.: 12/289,441

(22) Filed: Oct. 28, 2008

(30) Foreign Application Priority Data
Oct. 29, 2007 (DE) 10 2007 051 548.2

(51) Int. Cl. A61B 6/03

(52) U.S. Cl. 600/425; 378/8

(57) ABSTRACT

A method and a CT system are disclosed for measuring the perfusion in vessels and/or muscles of the heart (cardiac perfusion) in a patient. In at least one embodiment of the method the patient receives a contrast agent bolus, the patient is scanned for a scan period of a plurality of cardiac cycles in a scan field of a CT system controlled by the cardiac rhythm, a plurality of CT image data is reconstructed from projection data of a particular cardiac phase from respectively one cardiac cycle, and the temporal profile of the absorption values at at least one location in the heart is determined and displayed on the basis of a plurality of CT image data at successive times. At least one embodiment of the invention is distinguished by the fact that during the examination, the patient is repeatedly and alternately moved in opposite directions along a system axis of the CT system such that his cardiac region passes through the scan field at a cardiac phase range and the cardiac region is completely scanned spirally.

### PRIORITY STATEMENT

The present application hereby claims priority under 35 U.S.C. §119 on German patent application number DE 10 2007 051 548.2 filed Oct. 29, 2007, the entire contents of which is hereby incorporated herein by reference.

### FIELD

Embodiments of the invention generally relate to a method for measuring the perfusion in vessels and/or muscles of the heart (cardiac perfusion) in a patient. In at least one embodiment, the patient receives a contrast agent bolus, the patient being scanned for a scan period of a plurality of cardiac cycles in a scan field of a CT system controlled by the cardiac rhythm, and a plurality of CT image data are reconstructed from projection data of a particular cardiac phase from respectively one cardiac cycle. Subsequently, the temporal profile of the absorption values at at least one location in the heart are determined and displayed on the basis of a plurality of CT image data at successive times.

### BACKGROUND

A method for measuring the perfusion in the myocardium with the aid of a CT scan is widely known. For this purpose, a particular region of the heart is generally selected for the scan so that the volume scanned by the CT system represents the respective cardiac region of interest. Naturally, this requires the location of a cardiac region of diagnostic interest to be known prior to the examination, so that the temporal profile of a contrast agent wash-in can be subsequently determined and displayed by means of a circular scan of this region with the largest possible scan volume. However, in the case of CT systems which are used today having multi-row detectors with still relatively small scan volumes, an organ the size of the heart cannot yet be completely scanned within the scope of a stationary circular scan. Therefore, the problem of carrying out a perfusion measurement encompassing the whole heart remains.

### SUMMARY

In at least one embodiment of the invention, a method and a CT system are disclosed which increase the size of the scan volume, which was previously too small, so that the entire heart is scanned for a relatively long period of time such that image data which can also be used for a perfusion measurement is generated.

The inventors have recognized, in at least one embodiment, that it is possible to significantly increase the volume coverage of a CT scan within the scope of a perfusion examination of the heart by repeating the scan repeatedly and spirally while running in opposite directions. In the process, the direction of travel is reversed periodically and the same volume is repeatedly scanned spirally relative to the patient. In order to ensure that a perfusion signal generated from the CT images repeatedly recorded in a temporal sequence is acquired in the right phase, the patient couch can be moved while being controlled by an EKG. The heart beat directly before the diagnostic scan should be estimated prospectively from the preceding cardiac cycles.

Since the highest volume velocity is relevant for the diagnostic scan, it is particularly expedient, in at least one embodiment, if CT systems comprising a multiplicity of angularly offset emitter/detector systems are used for such a perfusion scan. For example, if two emitter/detector systems offset by 90° are used, then the volume velocity in the case of a 180° scan increases by a factor of 2. Thus, when the gantry rotates around by a quarter, a total scan region of 180° is achieved with the aid of the two emitter/detector combinations which are arranged on the gantry and are offset by an angle of 90°.

A further problem when determining the perfusion in the myocardium lies in the fact that, because of the relatively small lift of the absorption values of approximately 20 to 30 HU due to the applied contrast agent bolus, even only partial scan artifacts, which occur when image reconstructions based on semi-rotation data are used, lead to large measurement errors. This can be compensated for by calculating the reconstruction using the whole projection data collected over a complete rotation, that is to say over an angular range of 360°. However, in the context of a two-emitter/detector system, this does not mean that data from a complete rotation of the gantry must be used; rather it is sufficient to use data from a 270° rotation, with projection data from a total of 360° being made available by the emitter/detector systems offset by 90°. The temporal resolution of such CT systems is thus 3Tᵣₒₜ/4, where Tᵣₒₜ designates the rotation time of the scanner.

If different versions of CT systems with a number of x-ray sources arranged at an angular offset on the gantry are considered, then this of course yields different results with respect to the improved temporal resolution of these systems. The respective advantage of such systems with respect to the temporal resolution is inferred by a person skilled in the art directly by the spatial arrangement used for the emitter systems.

In accordance with at least one embodiment described above, the inventors propose a method for measuring the perfusion in vessels and/or muscles of the heart in a patient, the method comprising:
the patient receives a contrast agent bolus,
the patient is scanned for a scan period of a plurality of cardiac cycles in a scan field of a CT system comprising at least one radiation source controlled by the cardiac rhythm,
a plurality of CT image data is reconstructed from projection data of a particular cardiac phase from respectively one cardiac cycle, and
the temporal profile of the absorption values at at least one location in the heart is determined and displayed on the basis of a plurality of CT image data at successive times.

According to at least one embodiment of the invention, the method described above is supplemented by the fact that during the examination the patient is repeatedly and alternately moved in opposite directions along a system axis of the CT system such that his cardiac region passes through the scan field at a predetermined cardiac phase range and the cardiac region is completely scanned spirally.

As already described previously, it is particularly advantageous, in at least one embodiment, if the CT system comprises at least two radiation sources moved about the system axis, so that it is possible to improve the temporal resolution.

For example, if a CT system which has exactly two radiation sources which are arranged on a rotating gantry and are offset by an angle of 90° is used, then this results in an improvement of the temporal resolution relating to a semi-rotation scan by a factor of 2. Furthermore, image projection data from both radiation sources can be obtained over an angular range of altogether 360°, while the gantry only has to rotate through 270° and corresponding image data is reconstructed without partial rotation artifacts occurring.

If, alternatively, a CT system with exactly three radiation sources which are arranged on a rotating gantry and are offset by an angle of 120° is used in at least one embodiment, this results in a correspondingly more favorable temporal resolution; however, for this purpose complementary projections must be used for a semi-rotation reconstruction. However, such CT systems have relatively large problems correcting scattered radiation.

Furthermore, it is advantageous in at least one embodiment if the motion of the patient, or the patient table on which the patient lies, relative to the scan field of the CT system is controlled such that, during each passage of the cardiac region through the scan field, the relative velocity of the patient table to the scan field is constant. Under these circumstances, the CT system can be operated in a normal, standard data acquisition mode and the reconstructions can likewise be carried out without having to particularly take into account a possibly changing scan velocity.

Furthermore, it is also expedient if the motion of the patient relative to the scan field of the CT system is controlled and triggered by the cardiac rhythm signals such that in each case the cardiac region passes through the scan field during the predetermined cardiac phase range. In order to achieve this, it is necessary to predict, on the basis of one or more previously measured cardiac cycle periods, what the optimum time is for the cardiac region entering the scan field, and the velocity of the patient table, or also the acceleration of the table, must be adapted correspondingly, so that the constant relative velocity of the patient table is achieved at the right time and at the right position of the scan field. If these parameters are matched to one another, the patient and his cardiac region pass through the scan field with a high constant velocity, preferably during a rest phase of the heart. After reaching the end of the cardiac region, an optimum time at which the patient table is to be moved through the scan field again in the opposite direction is again predicted, based on the previous measurements of the cardiac frequency, and the reversal acceleration of the patient table is controlled correspondingly.

It is furthermore advantageous, in at least one embodiment, if the radiation source is active only when the cardiac region of the patient is in the scan field of the CT system. This minimizes the dosage given to the patient. In this context it should also be mentioned that it is of course ideal to record an orientation scan or topogram prior to carrying out the method of at least one embodiment, in order to determine the precise location and size of the cardiac region, so that the subsequent control can be optimally adapted to the actual position of the heart.

As an alternative to a 100% switching on and off of the radiation source, it is also possible to modulate the radiation source with respect to its dosage, so that the maximum dosage is only emitted when the cardiac region of the patient is in the scan field of the CT system.

Furthermore, it is expedient if the relative motion of the patient table, controlled by the cardiac rhythm, is triggered by the cardiac rhythm signals of an EKG connected to the patient. By way of example, it is possible in this case that the typical and easily extracted R wave of a concurrent EKG is used to adapt the relative motion of the patient table to the respective velocity of the heart.

Alternatively, it is also possible that the signal of a pressure-pulse sensor connected to a patient is used instead of an EKG signal in order to synchronously trigger the relative motion of the patient table with the cardiac rhythm signals.

In the process, it is also proposed that, in order to control the relative motion of the patient table, the cardiac rhythm is determined in each case on the basis of at least one preceding cardiac cycle, and the time of entering the predetermined cardiac phase, at which the cardiac region is to pass through the scan field, is predicted.

Furthermore, it is proposed that, at the reversal positions of the patient table, the cardiac region of the patient lies outside of the scan field. This therefore means that, at the moment of the motion reversal, the scan field is above or below the cardiac region. This gives enough opportunity for the acceleration phase of the table to attain a velocity which is as constant as possible when the cardiac region passes through. However, in this case it is also expedient that the distance of the reversal position from the cardiac region is selected so that it is not too great, so that the motion rhythms follow each other in the quickest succession possible, so that the heart is scanned with a sufficiently high frequency, that is to say the time between the individual scans is as short as possible.

Should the heart be scanned over a relatively long period of time, it is possible that the time between the individual scans of the particular cardiac phase changes due to changes in the cardiac frequency or intermittently occurring extrasystoles of the patient. Such a change needs to be taken into account when determining the temporal profile of the absorption values, that is to say the perfusion curve, so that such measurement values which are not equidistant temporally are compensated for by interpolation.

In principle, it is possible to carry out the method according to at least one embodiment of the invention using conventional CT systems which for scanning comprise one or more mechanically rotating x-ray tubes with opposing multirow detectors attached to a rotating gantry.

However, for a particularly high rotational velocity, it can be advantageous to use for scanning a stationary x-ray tube system with at least one multirow detector arranged on a rotating gantry, or else to use a stationary x-ray tube system with a likewise stationary multirow detector which surrounds the system axis through 360°.

The use of such stationary x-ray tube systems, which are controlled, for example, by circulating laser beams or electronically triggered cathode sections, makes it possible to achieve a substantially lower rotation time and thus achieve an improved temporal resolution of the system.

In addition to the method according to at least one embodiment of the invention described above, the inventors also propose a CT system in at least one embodiment with at least one emitter/detector system for scanning a patient and a control and computational unit with a memory for a computer program code, wherein the computer program code is stored in the memory and can execute the above-described invention steps of the method according to at least one embodiment of the invention during operation.

### BRIEF DESCRIPTION OF THE DRAWINGS

In the following text, embodiments of the invention will be described in more detail with reference to the figures, with only those features required for understanding embodiments of the invention being illustrated. In the process, the following reference symbols and abbreviated designations are used: 1: CT system; 2: first x-ray tube; 3: first multirow detector; 4: second x-ray tube; 5: second multirow detector; 6: gantry housing; 7: patient; 8: patient table; 9: system axis; 10: control and computational unit; 11: contrast agent injector; 12: EKG line; 13: displacement of the patient table with time; 13.1 to 13.4: linear motion; 14: EKG signal; 15: back-calculation of the optimum start of the linear motion phase; 16: velocity of the patient table with time; 17: acceleration of the patient table with time; 18.1 to 18.4: periods of linear motion; A: scan field; a(t): acceleration of the patient table; H: displacement across the cardiac region; Prg₁-Prgₙ: computer programs; s(t): displacement of the patient table; v(t): velocity of the patient table.

In detail:

FIG. 1 shows a CT system for carrying out the method according to an embodiment of the invention, and

FIG. 2 shows a temporally synchronized displacement/time, velocity/time and acceleration/time diagram.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

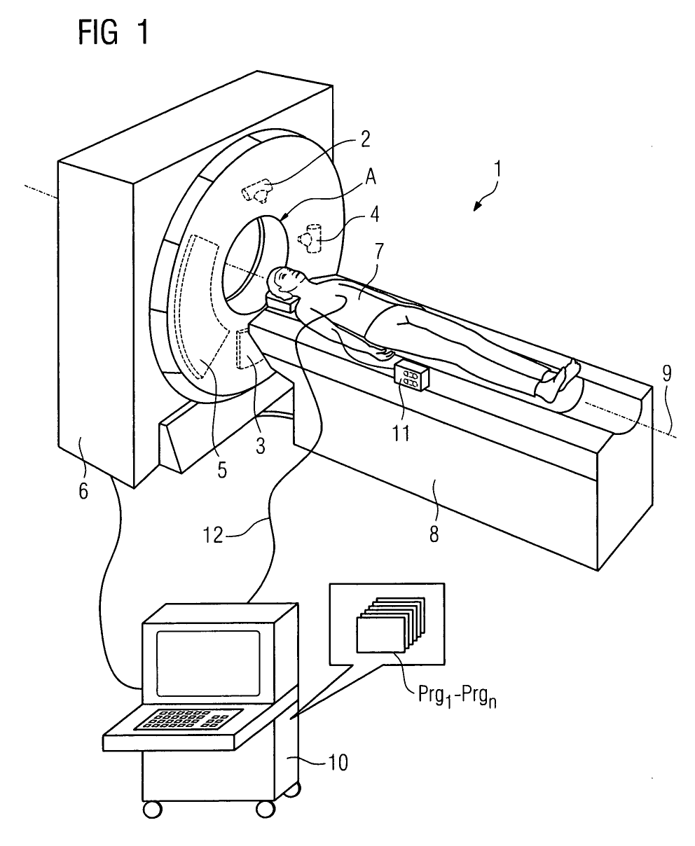

FIG. 1 shows an example conventional CT system 1 with two emitter/detector systems arranged on a gantry. The two emitter/detector systems are housed on a gantry (not explicitly shown) in the gantry housing 6 and comprise a first x-ray tube 2 with a first opposing multirow detector 3 and a second x-ray tube 4 with a second opposing multirow detector 5. A scan field A is generated between the x-ray tubes and the detectors by the interaction between the two x-ray tubes 2 and 4 and their opposing detectors 3 and 5 and, for the purposes of the scan, a patient 7 can be pushed through this scan field with the aid of a patient table 8 which can be displaced along the system axis 9. Due to the rotation of the two emitter/detector systems 2, 3 and 4, 5, and the relative motion of the patient 7 along the system axis 9, this results in a spiral scan relative to the patient 7 pushed through the scan field A.

The entire system is controlled with the aid of computer programs Prg₁ to Prgₙ, present in the memory of the control and computational unit 10 and executed during operation. The control and computational unit 10 is connected to the actual CT system via data and control lines so that the motion and dosage of the x-ray tubes, and the motion of the patient table 8, can be influenced in the desired way by this control and computational unit 10. Furthermore, the data collected by the two detector systems 3 and 5 are transferred to the control and computational unit 10 via corresponding lines. In addition, the control and computational unit 10 also comprises measurement systems which can record the potential curves of the heart via an EKG line 12, so that, in the presently illustrated case, an integrated EKG is present in the control and computational unit 10 which can correspondingly evaluate the measured EKG with the aid of the programs located therein, by means of which programs the triggering of motion of the patient table 8 according to an embodiment of the invention can be carried out.

To carry out the examinations of the patient according to an embodiment of the invention, it is furthermore necessary to inject the patient 7 at a particular time with a contrast agent bolus. This is effected by a contrast agent injector 11, which can likewise be controlled by the control and computational unit 10, or else can be operated independently.

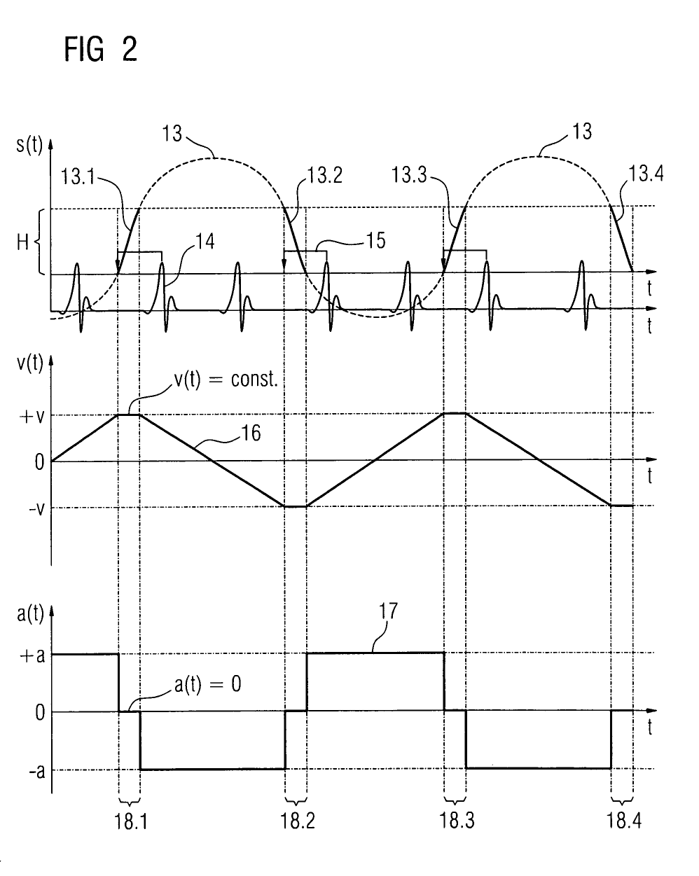

If the method according to an embodiment of the invention is effected with the aid of the control and computational unit 10, and the computer programs Prg₁ to Prgₙ integrated therein, this results in a temporal sequence of the motion of the patient table as illustrated in FIG. 2. The latter shows three motion diagrams over time, arranged one above the other. The displacement 13 of the patient table is illustrated in the first, top diagram, with the time t being plotted along the abscissa, and the displacement s(t) being plotted on the ordinate.

In the diagram lying below, the corresponding time is once again plotted on the abscissa, and the velocity v(t), that is to say the first derivative of the displacement s(t), is illustrated. Below that, the associated acceleration a(t) of the patient table is again plotted, likewise over the same temporal coordinate on the abscissa.

Additionally, the first, top diagram also shows a measured EKG signal 14 over the same temporal axis, so that the motion of the patient table relative to the heart motion becomes visible in FIG. 2. The motion curve 13 has a sinusoidal shape, which also illustrates the zigzag motion of the patient table, with linear path sections 13.1 to 13.4 being illustrated between the upper and lower reversal positions and during which the patient on the patient table and his cardiac region moves through the scan field of the CT system. The spatial arrangement of the cardiac region is characterized by the reference symbol H. To clarify the situation with regard to velocity and acceleration at a respective time, the diagrams lying below illustrate the velocity profile 16 of the patient table and the acceleration profile 17 of the patient table over the same temporal axis. It can be recognized in each case that, in the region of the motion of the patient table through the cardiac region H, on the one hand, the velocity is constant, and, on the other hand, the acceleration naturally has a value of zero.

Should it be intended that the motion of the patient or the patient table is now synchronized with the heart beat so that the patient table in each case passes through the scan field A with the cardiac region at precisely the right times 18.1 to 18.4, it is necessary to undertake a prediction with respect to the duration of the cardiac cycles in order to control the patient table at its reversal in such a manner that, on the one hand, the cardiac region enters the scan field A at precisely the right time in the cardiac phase and, on the other hand, the patient table has at this time also attained a linear velocity which is sufficient to completely scan the cardiac region during the predetermined cardiac phase. Of course, the rotational speed of the gantry has to be taken into account so that the scan is completed without gaps.

It is to be understood that the abovementioned features of embodiments of the invention can be used not only in the respectively specified combination but also in other combinations and on their own, without departing from the scope of the invention.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDS; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method, comprising:
scanning a patient, after receipt of a contrast agent bolus, for a scan period of a plurality of cardiac cycles of a heart in the patient in a scan field of a CT system including at least one radiation source controlled by cardiac rhythm;
reconstructing a plurality of CT image data from projection data of a cardiac phase from respectively one cardiac cycle; and
determining a temporal profile of absorption values at at least one location in the heart and displaying the temporal profile on the basis of a plurality of CT image data at successive times, wherein, during the scanning, the patient is repeatedly and alternately moved relative to the scan field of the CT system in opposite directions along a system axis of the CT system such that a cardiac region of the patient passes through the scan field at a cardiac phase range and such that the cardiac region is completely scanned spirally.

2. The method as claimed in claim 1, wherein the CT system includes at least two radiation sources moved about the system axis.

3. The method as claimed in claim 2, wherein exactly two radiation sources, arranged on a rotating gantry and offset by an angle of 90°, are used.

4. The method as claimed in claim 3, wherein projection data from both radiation sources over projection angles totaling 360° from a single 270° rotation of the gantry are used for each reconstruction.

5. The method as claimed in claim 2, wherein exactly three radiation sources, arranged on a rotating gantry and offset by an angle of 120°, are used.

6. The method as claimed in claim 5, wherein projection data from the three radiation sources over projection angles totaling 360° from a single 120° rotation of the gantry are used for each reconstruction.

7. The method as claimed in claim 1, wherein motion of the patient relative to the scan field of the CT system is controlled such that, during each passage of the cardiac region through the scan field, the relative velocity of the patient table to the scan field is constant.

8. The method as claimed in claim 1, wherein motion of the patient relative to the scan field of the CT system is controlled and triggered by the cardiac rhythm signals such that, in each case, the cardiac region passes through the scan field during the cardiac phase range.

9. The method as claimed in claim 1, wherein the at least one radiation source is active only when the cardiac region of the patient is in the scan field of the CT system.

10. The method as claimed in claim 1, wherein the at least one radiation source is modulated with respect to its dosage and only emits the maximum dosage when the cardiac region of the patient is in the scan field of the CT system.

11. The method as claimed in claim 1, wherein the relative motion of the patient table, controlled by the cardiac rhythm, is triggered by the cardiac rhythm signals of an EKG connected to the patient.

12. The method as claimed in claim 1, wherein the relative motion of the patient table, controlled by the cardiac rhythm, is triggered by the cardiac rhythm signals of a pressure-pulse sensor connected to the patient.

13. The method as claimed in claim 1, wherein, in order to control the relative motion of the patient table, the cardiac rhythm is determined; in each case, based on at least one preceding cardiac cycle, and the time of entering the predetermined cardiac phase, at which the cardiac region is to pass through the scan field, is predicted.

14. The method as claimed in claim 1, wherein, at the reversal positions of the patient table, the cardiac region of the patient lies outside of the scan field.

15. The method as claimed in claim 1, wherein measurement values, which are not equidistant, temporally are compensated for by interpolation when determining the temporal profile of the absorption values.

16. The method as claimed in claim 1, wherein mechanically rotating x-ray tubes with opposing multi-row detectors are used for scanning.

17. The method as claimed in claim 1, wherein a stationary x-ray tube system with at least one multirow detector arranged on a rotating gantry is used for scanning.

18. The method as claimed in claim 1, wherein a stationary x-ray tube system with a stationary multirow detector, which surrounds the system axis through 360°, is used for scanning.

19. A CT system, comprising:
at least one emitter/detector system to scan a patient; and
a control and computational unit including a memory, the memory storing a computer program code to execute the method as claimed in claim 1 during operation.

20. The method as claimed in claim 3, wherein motion of the patient relative to the scan field of the CT system is controlled such that, during each passage of the cardiac region through the scan field, the relative velocity of the patient table to the scan field is constant.

21. The method as claimed in claim 3, wherein motion of the patient relative to the scan field of the CT system is controlled and triggered by the cardiac rhythm signals such that, in each case, the cardiac region passes through the scan field during the cardiac phase range.

22. The method as claimed in claim 5, wherein motion of the patient relative to the scan field of the CT system is controlled such that, during each passage of the cardiac region through the scan field, the relative velocity of the patient table to the scan field is constant.

23. The method as claimed in claim 5, wherein motion of the patient relative to the scan field of the CT system is controlled and triggered by the cardiac rhythm signals such that, in each case, the cardiac region passes through the scan field during the cardiac phase range.

24. The method as claimed in claim 3, wherein mechanically rotating x-ray tubes with opposing multi-row detectors are used for scanning.

25. The method as claimed in claim 5, wherein mechanically rotating x-ray tubes with opposing multi-row detectors are used for scanning.

26. A CT system, comprising:
at least one emitter/detector system to scan a patient, after receipt of a contrast agent bolus, for a scan period of a plurality of cardiac cycles of a heart in the patient in a scan field of a CT system including at least one radiation source controlled by cardiac rhythm;
means for reconstructing a plurality of CT image data from projection data of a cardiac phase from respectively one cardiac cycle; and
means for determining a temporal profile of absorption values at at least one location in the heart and displaying the temporal profile on the basis of a plurality of CT image data at successive times, wherein, during the scanning, the patient is repeatedly and alternately moved relative to the scan field of the CT system in opposite directions along a system axis of the CT system such that a cardiac region of the patient passes through the scan field at a cardiac phase range and such that the cardiac region is completely scanned spirally.

27. A computer readable medium including program segments for, when executed on a computer device, causing the computer device to implement the method of claim 1.
